(19) United States
(12) Patent Application Publication
(10) Pub. No.: US 2005/0181456 A1
(43) Pub. Date: Aug. 18, 2005

(54) Methods for determining the amount of peptide YY in blood

(75) Inventors: Charles Arthur Foerder, Seattle, WA (US); Conor J. MacEvilly, Seattle, WA (US)

Correspondence Address: Nastech Pharmaceutical Company Inc., 3450 Monte Villa Parkway, Bothell, WA, 98021-8906, US

(73) Assignee: Nastech Pharmaceutical Company Inc.

(21) Appl. No.: 11/054,245

(22) Filed: Feb. 9, 2005

Related U.S. Application Data
(60) Provisional application No. 60/543,111, filed on Feb. 9, 2004.

(51) Int. Cl. G01N 33/53

(52) U.S. Cl. 435/7.1

(57) ABSTRACT

A method for determining the total amount of a Y2 receptor-binding such as PYY in a blood sample. Bound PYY is first freed from components in the blood and proteinaceous material in the blood precipitated preferably by the addition of an organic solvent miscible in water such as ethanol. The PYY remains in solution in a supernatant liquid. The supernatant liquid containing the PYY is removed and the total amount of PYY contained therein is determined preferably by means of an radioimmunoassay.

This claims benefit under 35 U.S.C. §119 (e) of U.S. Provisional Application No. 60/543,111, filed Feb. 9, 2004, the entire contents of which are incorporated herein by reference.

### BACKGROUND OF THE INVENTION

The teachings of all the references cited in the present specification are incorporated in their entirety by reference.

Obesity and its associated disorders are common and very serious public health problems in the United States and throughout the world. Upper body obesity is the strongest risk factor known for type-2 diabetes mellitus, and is a strong risk factor for cardiovascular disease. Obesity is a recognized risk factor for hypertension, arteriosclerosis, congestive heart failure, stroke, gallbladder disease, osteoarthritis, sleep apnea, reproductive disorders such as polycystic ovarian syndrome, cancers of the breast, prostate, and colon, and increased incidence of complications of general anesthesia. It reduces life-span and carries a serious risk of co-morbidities above, as well disorders such as infections, varicose veins, acanthosis nigricans, eczema, exercise intolerance, insulin resistance, hypertension hypercholesterolemia, cholelithiasis, orthopedic injury, and thromboembolic disease. Obesity is also a risk factor for the group of conditions called insulin resistance syndrome, or “Syndrome X.”

It has been shown that certain peptides that bind to the Y2 receptor when administered peripherally to a mammal induce weight loss. The Y2 receptor-binding peptides are neuropeptides that bind to the Y2 receptor. Neuropeptides are small peptides originating from large precursor proteins synthesized by peptidergic neurons and endocrine/paracrine cells. Often the precursors contain multiple biologically active peptides. There is great diversity of neuropeptides in the brain caused by alternative splicing of primary gene transcripts and differential precursor processing. The neuropeptide receptors serve to discriminate between ligands and to activate the appropriate signals. Peptide YY is a neuropeptide that binds to the Y2 receptor and is currently under development as an anti-obesity drug.

It has been shown that a 36 amino acid peptide called Peptide YY(1-36) [PYY(1-36)] [YPIKPEAPGEDASPEELNRYYASLRHYLNLVTRQRY, SEQ ID NO.: 1]. when administered peripherally by injection to an individual produces weight loss and thus can be used as a drug to treat obesity and related diseases, Morley, J. Neuropsychobiology 21:22-30 (1989). It was later found that to produce this effect PYY bound to a Y2 receptor, and the binding of a Y2 agonist to the Y2 receptor caused a decrease in the ingestion of carbohydrate, protein and meal size, Leibowitz, S. F. et al. Peptides, 12: 1251-1260 (1991). An alternate molecular form of PYY is PYY(3-36) IKPEAPGEDASPEELNRYYASLRHYLNLVTRQRY [SEQ ID NO.: 2], Eberlein, Eysselein et al. Peptides 10: 797-803, 1989). This fragment constitutes approximately 40% of total PYY-like immunoreactivity in human and canine intestinal extracts and about 36% of total plasma PYY immunoreactivity in a fasting state to slightly over 50% following a meal. It is apparently a dipeptidyl peptidase-IV (DPP4) cleavage product of PYY. PYY3-36 is reportedly a selective ligand at the Y2 and Y5 receptors, which appear pharmacologically unique in preferring N-terminally truncated (i.e. C-terminal fragments of) NPY analogs. It has also been shown that a PYY fragment having only residues 22-36 will still bind to the Y2 receptor. Hereinafter the term PYY refers to full-length PYY and any fragment of PYY that binds to a Y2 receptor.

However, to successfully develop a drug, one must be able to measure the total blood serum level of PYY. Thus, there is a need to develop improved methods for determining the total concentration of PYY in the blood serum.

### BRIEF DESCRIPTION OF THE DRAWINGS

FIG. 1 shows a standard curve for a radioimmunassay for determining the concentration of PYY in a sample of blood.

FIG. 2 shows the results of the determination of the concentration of PYY in blood from a number of individuals who were administered PYY intranasally at varying doses.

### DESCRIPTION OF THE INVENTION

The present invention fills this need by providing for a method for measuring an amount of a PYY in the blood serum comprised of comparing formulations to establish bioequivalence of a reference formulation and a test formulation of PYY. The assay is comprised of the following steps:
obtaining an aliquot of blood;
extracting PYY from cellular matrix present in the blood that binds PYY; and
measuring the concentration of PYY in said aliquot

Preferably the cellular components in the blood are removed first to produce an aliquot of plasma. An organic solvent, preferably alcohol, is added to the plasma resulting in the precipitation of proteins present in the plasma thus releasing the PYY. The precipitated proteins are removed from the plasma. The amount of PYY in the plasma can then be determined using standard antibody based competitive assays such as radioimmunoassay, enzyme-linked immunoassay, fluorescent immunoassay or chemiluminescent immunoassay.

The current invention is based upon the discovery that a portion of PYY present in plasma is bound to a plasma matrix and that a portion of the PYY bound to the matrix can be freed into solution by the precipitation of the matrix. This precipitation can be done using organic solvents such as ethanol, acetone, methanol or propanol, by salting out of the protein using standard salts like ammonium sulfate or by using organic polymers such as polyethylene glycol.

In doing an immunoassay a standard curve is first generated in which labeled PYY is placed in a number of tubes, each tube containing a different amount of labeled PYY. The concentration of PYY present in a tube then correlates with the amount of signal given off by the solution, the signal being radioactivity, flourescence, chemiluminescence or intensity of color in the solution. In a preferred embodiment, the immunoassay is a radioimmunoassay and the PYY is labeled with radioactive isotope such as ¹²⁵Iodine (¹²⁵I). The immunoassay to determine the concentration of PYY is an aliquot of plasma is a competitive assay in which a amount of antibody that binds to PYY is added in excess to the aliquot of plasma. Labeled PYY is then added to the plasma in an amount that would bind to the antibody initially added to the aliquot of plasma. An antibody that binds to the original antibody is then added to the plasma resulting in the precipitation of the PYY/antibody complex, which is then separated from the solute. The amount of label remaining in the solute reflects the amount of unbound, labeled PYY is remaining in the solute. By comparing the amount of labeled PYY remaining in solute from the amount of PYY originally added to the plasma to a standard curve, one can then determine the concentration of PYY originally present in the aliquot of plasma. If all of the labeled PYY is present in solution, then the concentration of the PYY in aliquot of plasma is too high to make an accurate determination of the amount of the PYY present in the plasma. The plasma should then be diluted, preferably using so-called stripped plasma from which all of the PYY has been removed.

The present invention is based upon the discovery that the total concentration PYY present in the blood serum cannot not be measured simply by doing a radioimmunoassay of the a sample of the serum. Such a prior art radioimmunoassay has been described by Grandt, D. et al., Regulatory Peptides, 51: 151-159 (1994). The present invention is based upon the discovery that a substantial amount of PYY is bound to other components of the blood serum, and is not accessible to the prior art radioimmunoassay. Thus, erroneous concentrations of PYY result when one uses the prior art assay because the total amount of PYY is not measured, only the free PYY.

The present invention fills this need by providing for an assay that measures the entire amount of a Y2 receptor-binding peptide, such as PYY, in a sample or aliquot of blood. The method for determining the total amount of a Y2 receptor-binding peptide, in particular, PYY comprised of obtaining an aliquot of blood, preferably plasma, extracting the Y2 receptor-binding peptide from components in the blood to which the peptide may be bound to facilitate a more complete isolation of the total amount of the Y2 receptor-binding peptide, and measuring the amount of the Y2 receptor-binding peptide recovered from the blood sample. In the preferred embodiment, the Y2 receptor-binding peptide is PYY and the method for determining the amount of the free PYY is an immunoassay.

In the preferred embodiment, the bound PYY is freed by using an ethanol extraction. A organic solvent miscible in water such as methanol, ethanol, propanol or butanol, preferably ethanol, is added to an aliquot of plasma such that the proteinaceous material present in the sample precipitates leaving behind in solution PYY that was bound to the proteinaceous matrix. The sample is centrifuged resulting in a solid proteinaceous pellet at the bottom of the centrifuge tube, and the PYY remains in the supernatant liquid. The supernatant liquid containing the PYY is then removed and placed in a second container. The supernatant liquid is then dried, preferably under vacuum, leaving behind the PYY. The PYY is then resuspended in an immunoassay buffer and the total amount of PYY present is determined using standard immunoassay techniques as shown in Examples 1-3.

The present invention is further comprised of a method for measuring an amount of a Y2 receptor-binding present in the blood serum comprised of comparing formulations to establish bioequivalence of a reference formulation and a test formulation of a Y2 Receptor Binding compound. The assay is comprised of the following steps:
a. Administering the reference and test formulation sequentially to a group of subjects and obtaining blood samples at intervals after said administration;
b. obtaining a solvent extracted plasma sample from said blood samples;
c. measuring the concentration of Y2 Receptor Binding compound in said samples and determining the pharmacokinetic parameters, including Cₘₐₓ and AUC, for the reference and test formulations;
d. comparing the test and reference formulation pharmacokinetic parameters to determine bioequivalency.

To provide a better understanding of the present invention, the following definitions are provided:

### Peptide YY Agonists

As used herein, “PYY” refers to PYY(1-36) in native-sequence or in variant form, as well as derivatives, fragments, and analogs of PYY from any source, whether natural, synthetic, or recombinant. The PYY must be comprised at least the last 15 amino acid residues or analogoues thereof of the PYY sequence, PYY(22-36) (SEQ ID NO: 3). Other PYY peptides, which may be used are PYY(1-36) (SEQ ID NO: 1) PYY(3-36) SEQ ID NO: 2) PYY(4-36 )(SEQ ID NO:4) PYY(5-36) (SEQ ID NO: 5), PYY(6-36) (SEQ ID NO:6), PYY(7-36) (SEQ ID NO:7) PYY(8-36) (SEQ ID NO: 8), PYY9-36 (SEQ ID NO: 9) PYY(10-36) (SEQ ID NO: 10), PYY(1 1-36) (SEQ ID NO: 11), PYY(12-36) (SEQ ID NO: 12), PYY(13-36) (SEQ ID NO:13), PYY(14-36) (SEQ ID NO: 14), PYY(15-36) (SEQ ID NO: 15), PYY(16-36) (SEQ ID NO: 16), PYY(17-36) (SEQ ID NO: 17), PYY(18-36) (SEQ ID NO: 18), PYY(19-36) (SEQ ID NO: 19), PYY(20-36) (SEQ ID NO: 20) and PYY(21-36) (SEQ ID NO: 21). These peptides typically bind to the Y receptors in the brain and elsewhere, especially the Y2 and/or Y5 receptors. Typically these peptides are synthesized in endotoxin-free or pyrogen-free forms although this is not always necessary.

Other PYY peptides include those PYY peptides in which conservative amino acid residue changes have beem made, for example, site specific mutation of a PYY peptide including [Asp¹⁵] PYY(15-36) (SEQ ID NO: 22), [Thr¹³] PYY(13-36) (SEQ ID NO: 23), [Val¹²] PYY(12-36)(SEQ ID NO: 24), [Glu¹¹] PYY(11-36) (SEQ ID NO: 25), [Asp¹⁰] PYY(10-36) (SEQ ID NO: 26), [Val⁷] PYY(7-36) (SEQ ID NO: 27), [Asp⁶]PYY(6-36) (SEQ ID NO: 28), [Gln⁴] PYY(4-36) (SEQ ID NO: 29), [Arg⁴] PYY(4-36) (SEQ ID NO: 30), [Asn⁴] PYY(4-36) (SEQ ID NO: 31), [Val³] PYY(3-36) (SEQ ID NO: 32) and [Leu³] PYY(3-36) (SEQ ID NO: 33). Other PYY peptides include those peptides in which at least two conservative amino acid residue changes have been made including [Asp¹⁰, Asp¹⁵] PYY(10-36) (SEQ ID NO: 34), [Asp⁶, Thr¹³] PYY(6-36) (SEQ ID NO: 35), [Asn⁴, Asp¹⁵] PYY(4-36) (SEQ ID NO: 36) and [Leu³, Asp¹⁰] PYY(3-36) (SEQ ID NO: 37). Also included are analogues of a PYY for example those disclosed in U.S. Pat. Nos. 5,604,203 and 5,574,010; Balasubramaniam, et al., Peptide Research 1: 32 (1988); Japanese Patent Application 2,225,497 (1990); Balasubramaniam, et al., Peptides 14: 1011, 1993; Grandt, et at., Reg. Peptides 51: 151, (1994); PCT International Application 94/03380, U.S. Pat. Nos. 5,604,203 and 5,574,010. These peptides typically bind to the Y receptors in the brain and elsewhere, especially the Y2 and/or Y5 receptors. Typically these peptides are synthesized in endotoxin-free or pyrogen-free forms although this is not always necessary.

PYY agonists include rat PYY (SEQ ID NO: 38) and the amino terminus truncated forms corresponding to the human, pig PYY (SEQ ID NO: 39) and the amino terminus truncated forms corresponding to the human and guinea pig PYY (SEQ ID NO: 40) and the amino terminus truncated forms corresponding to the human.

As used herein “peak concentration (Cₘₐₓ) of PYY in a blood plasma”, “area under concentration vs. time curve (AUC) of PYY in a blood plasma”, “time to maximal plasma concentration (tₘₐₓ) of PYY” are pharmacokinetic parameters known to one skilled in the art. Laursen et al., Eur. J. Endocrinology, 135: 309-315, 1996. The “concentration vs. time curve” measures the concentration of PYY in blood serum of a subject vs. time after administration of a dosage of Y2 receptor-binding peptide to the subject either by intranasal, intramuscular, subcutaneous, or other parenteral route of administration. “Cₘₐₓ” is the maximum concentration of PYY in the blood serum of a subject following a single dosage of PYY to the subject. “tₘₐₓ” is the time to reach maximum concentration of PYY in blood serum of a subject following administration of a single dosage of PYY to the subject.

As used herein, “area under concentration vs. time curve (AUC) of PYY in a blood plasma” is calculated according to the linear trapezoidal rule and with addition of the residual areas. A decrease of 23% or an increase of 30% between two dosages would be detected with a probability of 90% (type II error β=10%). The “delivery rate” or “rate of absorption” is estimated by comparison of the time (tₘₐₓ) to reach the maximum concentration (Cₘₐₓ). Both Cₘₐₓ and tₘₐₓ are analyzed using non-parametric methods. Comparisons of the pharmacokinetics of intramuscular, subcutaneous, intravenous and intranasal PYY administrations were performed by analysis of variance (ANOVA). For pair wise comparisons a Bonferroni-Holmes sequential procedure was used to evaluate significance. The dose-response relationship between the three nasal doses was estimated by regression analysis. P<0.05 was considered significant. Results are given as mean values±SEM.

The following examples are provided by way of illustration, not limitation.

### EXAMPLE 1

### Total Extraction Radioimmunoassay for the Determination of the Concentration of PYY in Plasma

1.0 Introduction:

A radioimmunoassay was developed to measure the concentration of Human Peptide YY(3-36) (hPYY) in human plasma. Approximately 1 mL of blood was drawn from the individual in a tube containing anticoagulant (EDTA) and protease inhibitor (aprotinin). The aliquot of blood was centrifuged and the plasma removed and placed in a tube with anticoagulant (EDTA) and protease inhibitor (aprotinin) and frozen. The assay was a four day process. Samples, controls, and standards were extracted in alcohol and dried on Day 1. All samples were reconstituted and mixed with a polyclonal rabbit antiserum directed against hPYY on Day 2. Iodinated hPYY was added on Day 3. Specific precipitating agents (Goat anti-Rabbit IgG and Normal Rabbit Serum) were added on Day 4. Bound tracer was separated from free tracer by centrifugation, and the bound tracer was counted in a gamma counter. Concentration was calculated by interpolation of a standard curve and assay performance was controlled with Quality Control samples.

2.0 Materials:

2.1 Peninsula PYY kit (Peninsula Laboratories, Cat. No. S-2043-0001)

2.2 Reagent Alcohol (Fisher Inc., Cat. No. A995-4) (or equivalent)

2.3 Stripped human plasma (with Lithium Heparin, fasted, pooled) Golden West Biologics Inc. (Cat. No., SD1020-H)

2.4 Ice Baths (Fisher, Cat No. 11-676-36) (or equivalent)

2.5 Disposable 10 mL pipets (Fisher Cat. No. 13-678-11E) (or equivalent)

2.6 Standard Synthetic Human PYY from (3-36) (Bachem Cat. No. H8585)

2.7 Distilled Water (Milli-Q Millipore, Cat. No. ZMQ56VFT1) (or equivalent)

2.8 Triton X-100 (Sigma, Cat. No. T-9284) (or equivalent)

2.9 Aprotinin (ICN Biomedicals Inc. Cat. No. 190779) (or equivalent)

2.10 12×75 mm tubes (Evergreen Scientific, Cat. No. 214-2023-010) (or equivalent)

2.11 12×75 mm tube caps (Evergreen Scientific, Cat. No. 300-2912-G20) (or equivalent)

2.12 1.5 mL microfuge tubes (Fisher, Cat. No. 05-402-25) (or equivalent)

3.0 Instruments:

3.1 Wallac WIZARD 1470 Automatic Gamma Counter (Perkin Elmer, Model No. 1470-002) (or equivalent)

3.2 Isotemp Basic Freezer, −70° C. (Kendro Laboratory Products, Model No. C90-3A31) (or equivalent)

3.3 CentriVap Concentrator (Labconco, Cat. No. 7810000) (or equivalent)

3.4 VX-2500 Multi-tube Vortexer (VWR, Cat. No. 58816-115) (or equivalent)

3.5 Marathon 21000R Centrifuge (Fisher, Cat. No. 04-977-21000R) (or equivalent)

3.6 Swinging bucket rotor (Fisher, Cat. No. 04-976-006) (or equivalent)

3.7 Motorized pipet-aid (Fisher, Cat. No. 13-681-15E) (or equivalent)

3.8 Eppendorf Micropipette
3.8.1 2 μL-20 μL (Fisher, Cat. No. 21-371-6) (or equivalent)
3.8.2 20 μL-200 μL (Fisher, Cat. No.21-371-10) (or equivalent)
3.8.3 100 μL-1000 μL (Fisher, Cat. No.21-371-13) (or equivalent)

3.9 Eppendorf Repeating Pipettor (Fisher, Cat. No. 21-380-9) (or equivalent)

3.10 Eppendorf Repeating Pipettor Combi-tips
3.10.1 2.5 mL (Fisher, Cat. No. 21-381-331) (or equivalent)
3.10.2 25 mL (Fisher, Cat. No. 21-381-115) (or equivalent)

3.11 Positive displacement pipet (Fisher, Cat. No. 21-169-10A) (or equivalent)

4.0 Procedure

Day 1

### Preparation of Samples Containing Known Amounts of PYY for the Preparation of a Standared Curve

4.1 All reagents and samples were thawed for the assay. Radioimmunoassay buffer (RIAB) was prepared to 1× concentration.

4.2 S A standard curve indicating the concentration in samples containing known amount of PYY was prepared using pooled human plasma from which all of the PYY was removed. The samples containing the known amount of PYY were prepared using a starting concentration of 12.8 μg/mL. Tubes were labeled as indicated and shown in Table 1 below.
4.2.1 990 μL of RIAB was added to a tube labeled O.
4.2.2 990 μL of pooled plasma stripped of PYY was added to a tube labeled A.
4.2.3 500 μL of pooled plasma stripped of PYY was added to tubes labeled B-H.
4.2.4 10 μL of solution having a concentration of 12.8 μg/mL of PYY Standard was added to tube O and the tube was vortexed.
4.2.5 1 0 μL of the solution from tube O was added to tube A and the tube was vortexed.
4.2.6 500 μL of the solution from tube A was added to tube B and the tube was vortexed.
4.2.7 500 μL of the solution from tube B was added to tube C and the tube was vortex.
4.2.8 500 μL of the solution from tube C was added to tube D and the tube was vortex.
4.2.9 500 μL of the solution from tube D was added to tube E and the tube was vortex.
4.2.10 500 μL of the solution from tube E was added to tube F and the tube was vortex.
4.2.11 500 μL of the solution from tube F was added to tube G and the tube was vortex.
4.2.12 500 μL of the solution from tube G was added to tube H and the tube was vortex.

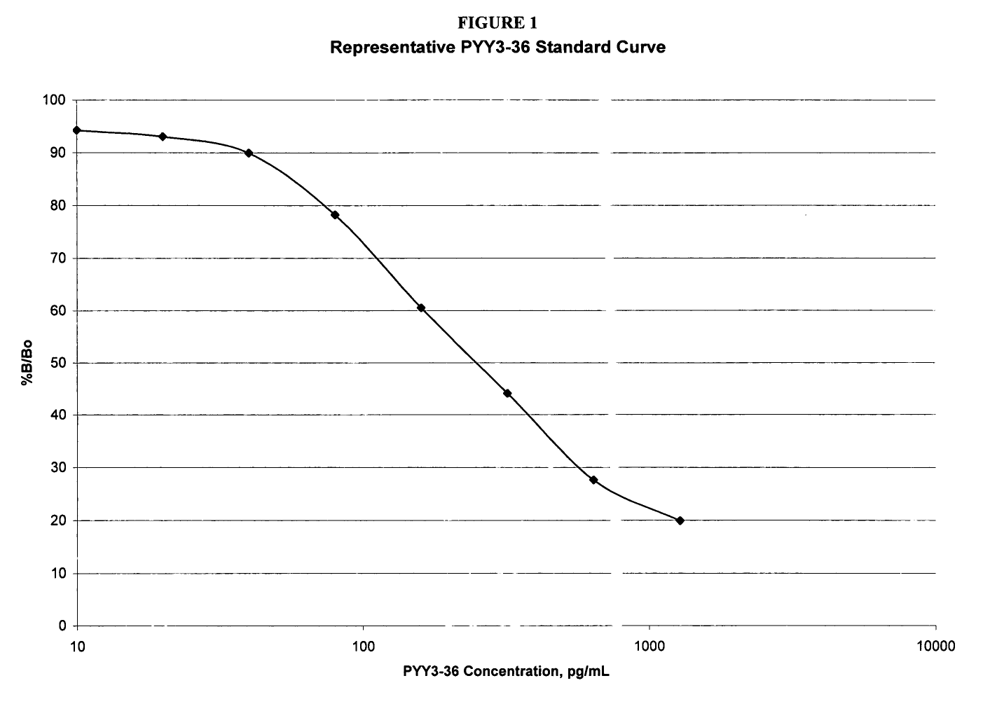

FIG. 1 shows the standard curve measuring the amount of PYY in solution in the standard solutions as described above.

Preparation and Measurement of PYY Present in Human Plasma Samples

Blood was drawn and centrifuged and the plasma was extracted using standard techniques.

4.3 The samples of unknown human plasma to be tested should be diluted using pooled human plasma stripped of PYY, if necessary.

4.4 1.2 mL of cold alcohol were added to empty tubes for NSB, TB, all Standards, QC samples, and human plasma samples to be tested.

4.5 400 μL of pooled human plasma stripped of PYY were added to NSB and TB tubes and vortexed.

4.6 400 μL of each prepared Standard sample from 4.2.5 to 4.2.8 were added to respective standard curve tubes H-A. Cap, Vortex.

4.7 400 μL of QC samples were added to respective tubes and the tubes were vortexed.

4.8 400 μL of each sample to be tested were added to its respective tube and vortexed

4.9 All samples were incubated on ice for 30-60 minutes.

4.10 The cold-trap switch on the Concentrator was turned on.

4.11 All tubes at were centrifuged at 3000 rpm, 4° C. for 15 minutes.

4.12 1.3 mL of supernatant from each sample were transferred to a new set of empty tubes.

4.13 The samples were placed in the Concentrator.

4.14 Samples were spun for two hours at 40° C., then at ambient temperature for a total of 5 hours or until dry.

4.15 The dried samples were removed from the concentrator, covered and stored overnight at 2-8° C.

Day 2

4.16 The dried tubes were removed from cooler.

4.17 100 μL of 4× RIA buffer concentrate were added to each tube.

4.18 100 μL of 0.6% TX100 were added to each tube, (Attachment #1) and vortexed for aminimum of 30 seconds to ensure all extracts are fully reconstituted.

4.19 All samples were incubated on ice for 30-60 minutes.

4.20 200 μL of distilled water were added to each tube and vortexed.

4.21 Transfer 100 μL of each sample extract to respective tube.
Note: non-specific binding (NSB), total bound (TB), total counts (TC), Standard Curve samples, and quality control samples (QC) are typically run in triplicate, requiring three tubes per sample. Human plasma samples many be tested in any variation (up to three replicates) depending on sample availability.

4.22 Rabbit anti-PYY was prepared as described in the Peninsula Laboratories kit insert.

4.23 100 μL of RIAB was added to each NSB tube.

4.24 200 μL RIAB was added to each TC tube.

4.25 100 μL Rabbit anti-PYY was added to all remaining tubes, and the tubes were vortexed, and covered with foil and stored overnight at 2-8° C.

Day 3

4.26 The tubes were removed from the 2-8° C. cooler.

4.27 ¹²⁵I-Peptide YY tracer was prepared as shown below in Attachment #2.

4.28 100 μL of prepared tracer were added to all tubes and the tubes were vortexed and overnight at 2-8° C.

Day 4

4.29 The tubes were removed from the 2-8° C. cooler.

4.30 Goat anti-Rabbit IgG serum (GARGG) and Normal Rabbit Serum (NRS) were prepared as described in the Peninsula Laboratories kit insert.

4.31 100 μL of GARGG were added to each tube (except TC tubes).

4.32 100 μL NRS were added to each tube (except TC tubes). Vortex.

4.33 The tubes were incubated for 90-120 minutes at room temperature.

4.34 500 μL RIAB were added to the tubes to be centrifuged immediately (except TCtubes), and the tubes were vortexed.
Note: 500 μL RIAB should be added to tubes just prior to centrifugation. Only add RIAB to the number of tubes that are ready to be centrifuged. 500 μL RIAB should be added to additional tubes when they are ready to be centrifuged.

4.35 The tubes containing 500 μL RIAB were centrifuged at 3000 rpm at 4° C., for 15 minutes. Do not centrifuge TC tubes.

4.36 Supernatant was aspirated and discarded from centrifuged tubes.

4.37 All the tubes were placed in racks and the emitted gamma radiation was determined using the Gamma counter.

5.0 Evaluation of Results

5.1 The following guidelines are applied to the identification and rejection of outliers in the assay. In order for a result to qualify as an outlier and not be included in the final calculation of results, all of the following conditions must be met.
5.1.1 QCs and unknown samples:
   5.1.1.1 % CV of all replicates must be great than 20%.
   5.1.1.2 There must be at least three results to evaluate.
   5.1.1.3 The difference between the suspected outlier and the result next closest in value must be greater than 20%.
   5.1.1.4 The difference between the high and low remaining results must be less than 20%.
5.1.2 Standard Curve samples:
   5.1.2.1 % CV of all replicates much be greater than 15%.
   5.1.2.2 There must be at least three results to evaluate.
   5.1.2.3 The difference between the suspected outlier and the result next closest in value must be greater than 15%.
   5.1.2.4 The difference between the high and low remaining results must be less than 15%.
      6.0 Assay Specifications

6.1 QC samples are prepared at the following concentrations. Two QC samples at each concentration are tested in an assay. Four of the six QC samples tested must be within the following ranges (±30% of nominal concentration). At least one of the two QCs tested at any concentration must be within range of the assay for data to be acceptable.
6.1.1 QC1 (100 pg/mL) 70-130 pg/mL
6.1.2 QC2 (200 pg/mL) 140-260 pg/mL

6.1.3 QC3 (500 pg/mL) 350-650 pg/mL

TABLE 1
PYY RIA Standard:

| Tube | |
|---|---|
| designation | Concentration of Standard |
| A | 1280 pg/mL |
| B | 640 pg/mL |
| C | 320 pg/mL |
| D | 160 pg/mL |
| E | 80 pg/mL |
| F | 40 pg/mL |
| G | 20 pg/mL |
| H | 10 pg/mL |

Attachment #1

| | |
|---|---|
| | 0.6% TX-100 |
| Reagent: | 0.6% TX-l00 |
| Materials: | Milli-Q Distilled Water |
| | TX-100 |
| Preparation: | 1) Measure 50 mL of Milli-Q Distilled Water |
| | 2) Add 300 μL of TX-100 using positive |
| | displacement pipet |
| | 3) Mix well. |
| Attachment #2 | |
| | ¹²⁵I-Peptide PYY Tracer |
| Reagent: | ¹²⁵I-Peptide PYY Tracer |
| Materials: | 1x RIA Buffer |
| | ¹²⁵I-Peptide PYY |
| Preparation: | 1) Reconstitute tracer with 1 mL of 1x RIA Buffer. |
| | 2) Measure the quantity of the tracer on the |
| | Gamma Counter. Transfer 10 μL of reconstituted |
| | tracer to a tube. Place it in a black |
| | rack for the Gamma Counter with Program #30 attached. |
| | 3) Place rack on the Gamma Counter with the |
| | Stop rack behind it. |
| | 4) Press ‘Start” to begin counting, |
| | then ‘E’ to view CPM results. |
| | 5) Determine amount of tracer (X μL) to prepare and |
| | RIAB (Y mL) needed as follows: |

$\begin{matrix}X μL = \frac{( 5 μL ) ( cpm value ) ( \# tubes + 10 )}{( cpm from stock solution )} \\ Y mL = ( 0.1 ) ( \# tubes + 10 )\end{matrix}$

| | |
|---|---|
| 6) | Combine X μL of ¹²⁵I-Peptide YY with Y mL of RIAB. |
| | Mix well. |

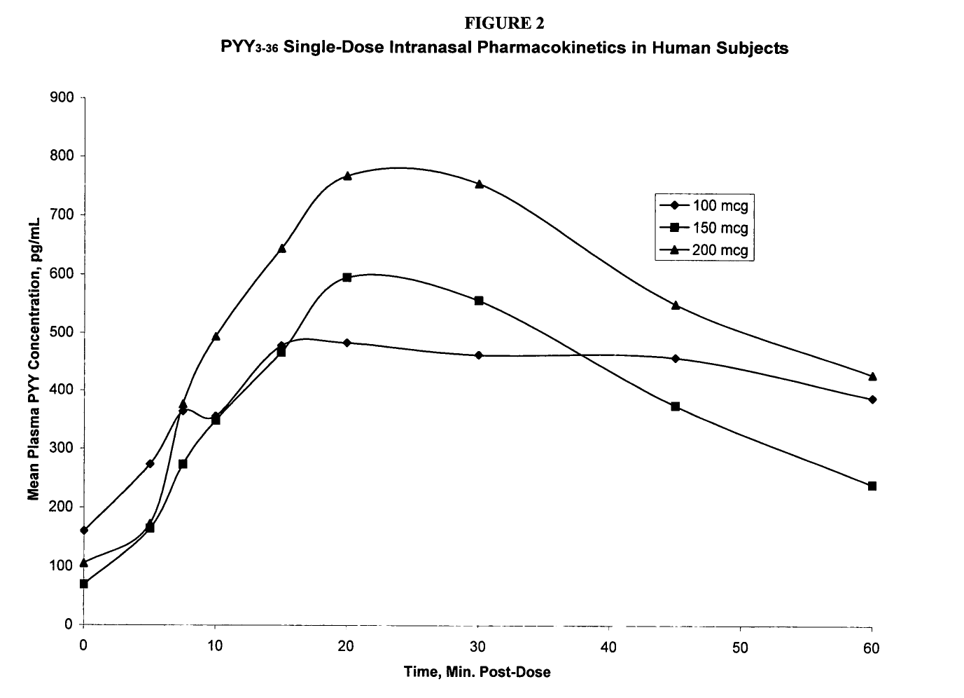

FIG. 1 shows the standard curve and FIG. 2 shows the result of a study in which the concentration of PYY in the blood was determined from individuals given different doses of PYY3-36, which was administered intranasally.

### EXAMPLE 2

4.0 Introduction:

A radioimmunoassay was developed to measure the concentration of Human Peptide YY 3-36 (hPYY) in rat plasma. Samples are collected with anticoagulant (EDTA) and protease inhibitor (aprotinin) and frozen. The assay was a four day process. Samples, controls, and standards are extracted in alcohol and dried on Day 1. All samples are reconstituted and mixed with a polyclonal rabbit antiserum directed against hPYY on Day 2. Iodinated hPYY is added on Day 3. Specific precipitating agents (Goat anti-Rabbit IgG and Normal Rabbit Serum) are added on Day 4. Bound tracer is separated from free tracer by centrifugation, and the bound tracer is counted in the gamma counter. Concentration is calculated by interpolation of a standard curve and assay performance is controlled with Quality Control samples.

5.0 Materials:

2.1 Peninsula PYY kit (Peninsula Laboratories, Cat. No. S-2043-0001)

2.3 Reagent Alcohol (Fisher, Cat. No. A995-4) (or equivalent)

2.5 Stripped rat plasma (Sprague Dawley with EDTA, fasted, pooled) Golden West Biologics Inc. (Cat. No., ABP2150-E)

2.6 Ice Baths (Fisher, Cat No. 11-676-36) (or equivalent)

2.5 Disposable 10 mL pipets (Fisher Cat. No. 13-678-11E) (or equivalent)

2.8 Standard Synthetic Human PYY (3-36) (Bachem Cat. No. H8585)

2.9 Distilled Water (Milli-Q Millipore, Cat. No. ZMQ56VFT1) (or equivalent)

2.8 Triton X-100 (Sigma, Cat. No. T-9284) (or equivalent)

2.9 Aprotinin (ICN Biomedicals Inc. Cat. No. 190779) (or equivalent)

2.10 12×75 mm tubes (Evergreen Scientific, Cat. No. 214-2023-010) (or equivalent)

2.11 12×75 mm tube caps (Evergreen Scientific, Cat. No. 300-2912-G20) (or equivalent)

2.12 1.5 mL microfuge tubes (Fisher, Cat. No. 05-402-25) (or equivalent)

6.0 Instruments:

6.1 Wallac WIZARD 1470 Automatic Gamma Counter (Perkin Elmer, Model No. 1470-002) (or equivalent)

6.2 Isotemp Basic Freezer, −70° C. (Kendro Laboratory Products, Model No. C90-3A31) (or equivalent)

6.3 CentriVap Concentrator (Labconco, Cat. No. 7810000) (or equivalent)

6.4 VX-2500 Multi-tube Vortexer (VWR, Cat. No. 58816-115) (or equivalent)

6.5 Marathon 21000R Centrifuge (Fisher, Cat. No. 04-977-21000R) (or equivalent)

6.6 Swinging bucket rotor (Fisher, Cat. No. 04-976-006) (or equivalent)

6.7 Motorized pipet-aid (Fisher, Cat. No. 13-681-15E) (or equivalent)

6.8 Eppendorf Micropipette
6.8.1 2 μL-20 μL (Fisher, Cat. No. 21-371-6) (or equivalent)
6.8.2 20 μL-200 μL (Fisher, Cat. No.21-371-10) (or equivalent)

6.8.3 100 μL-1000 μL (Fisher, Cat. No.21-371-13) (or equivalent)

6.9 Eppendorf Repeating Pipettor (Fisher, Cat. No. 21-380-9) (or equivalent)

6.10 Eppendorf Repeating Pipettor Combi-tips
6.10.1 2.5 mL (Fisher, Cat. No.21-381-331) (or equivalent)
6.10.2 25 mL (Fisher, Cat. No. 21-381-115) (or equivalent)

6.11 Positive displacement pipet (Fisher, Cat. No. 21-169-10A) (or equivalent)

4.0 Procedure

Day 1

4.38 Thaw necessary reagents and samples for the assay. Prepare RIA buffer to 1× concentration (RIAB) if sufficient amount is not available.

4.39 Prepare standard curve samples in pooled stripped rat plasma. Prepare as follows if using a starting concentration of 12.8 μg/mL.
4.39.1 Add 990 μL RIAB to tube O.
4.39.2 Add 990 μL pooled plasma to tube A.
4.39.3 Add 500 μL pooled plasma to tubes B-H.
4.39.4 Add 10 μL 12.8 μg/mL Standard to tube O. Vortex.
4.39.5 Add 10 μL solution from tube O to tube A. Vortex.
4.39.6 Add 500 μL solution from tube A to tube B. Vortex.
4.39.7 Add 500 μL solution from tube B to tube C. Vortex.
4.39.8 Repeat dilutions as in 4.2.7 through tube H. (See Diagram #1)

4.40 Dilute unknown rat plasma samples to be tested if necessary. Samples should be diluted in pooled stripped rat plasma. Each sample should have a final volume of 400 μL of neat or diluted sample.

4.41 Add 400 μL of pooled stripped rat plasma to NSB and TB tubes.

4.42 Add 400 μL of each prepared Standard sample from 4.2.5 to 4.2.8 to respective standard curve tubes H-A.

4.43 Add 400 μL of QC samples to respective tubes.

4.44 Add 1.2 mL of cold alcohol to tubes containing NSB, TB, all Standards, QC samples, and rat plasma samples to be tested. Cap, vortex.

4.45 Incubate all samples on ice for 30-60 minutes.

4.46 Turn on the cold-trap switch on the Concentrator.

4.47 Centrifuge all tubes at 3000 rpm, 4° C. for 15 minutes.

4.48 Transfer 1.3 mL of supernatant from each sample to a new set of empty tubes. Store in an ice bath or at 2-8° C. if not spun immediately.

4.49 Place samples in the Concentrator.

4.50 Samples should spin for two hours at 40° C., then at ambient temperature for a total of 5 hours or until dry.

4.51 Remove dried samples, cover and store overnight at 2-8° C.

Day 2

4.52 Remove the dried tubes from the 2-8° C. cooler.

4.53 Add 100 μL of 4× RIA buffer concentrate to each tube.

4.54 Add 100 μL of 0.6% TX100 to each tube. (Attachment #1) Vortex for a minimum of 30 seconds to ensure all extracts are fully reconstituted.

4.55 Incubate all samples on ice for 30-60 minutes.

4.56 Add 200 μL of distilled water to each tube. Vortex.

4.57 Transfer 100 μL of each sample extract to respective tube.
Note: NSB, TB, TC, Standard Curve samples, and QCs are typically run in triplicate, requiring three tubes per sample. Rat plasma samples many be tested in any variation (up to three replicates) depending on sample availability.

4.58 Prepare Rabbit anti-PYY as described in the Peninsula Laboratories kit insert.

4.59 Add 100 μL RIAB to each NSB tube.

4.60 Add 200 μL RIAB to each TC tube.

4.61 Add 100 μL Rabbit anti-PYY to all remaining tubes. Vortex.

4.62 Cover with foil and store overnight at 2-8° C.

Day 3

4.63 Remove the tubes from the 2-8° C. cooler.

4.64 Prepare ¹²⁵I-Peptide YY tracer (Attachment #2).

4.65 Add 100 μL of prepared tracer to all tubes. Cap and vortex.

4.66 Store overnight at 2-8° C.

Day 4

4.67 Remove the tubes from the 2-8° C. cooler.

4.68 Prepare Goat anti-Rabbit IgG serum (GARGG) and Normal Rabbit Serum (NRS) as described in the Peninsula Laboratories kit insert.

4.69 Add 100 μL GARGG to each tube (except TC tubes).

4.70 Add 100 μL NRS to each tube (except TC tubes). Vortex.

4.71 Incubate 90-120 minutes at room temperature.

4.72 Add 500 μL RIAB to tubes to be centrifuged immediately (except TC tubes). Vortex.
Note: 500 μL RIAB should be added to tubes just prior to centrifugation. Only add RIAB to the number of tubes that are ready to be centrifuged. 500 μL RIAB should be added to additional tubes when they are ready to be centrifuged.

4.73 Centrifuge tubes (containing 500 μL RIAB) at 3000 rpm at 4° C., for 15 minutes. Do not centrifuge TC tubes.

4.74 Aspirate supernatant from centrifuged tubes.

4.75 Place tubes in designated for counting on the Gamma counter and count the gamma radiation using standard techniques

7.0 Evaluation of Results

7.1 The following guidelines are applied to the identification and rejection of outliers in the assay. In order for a result to qualify as an outlier and not be included in the final calculation of results, all of the following conditions must be met. Outliers can be removed based on CPM or concentration results.
7.1.1 There must be at least three results to evaluate.
7.1.2 Removal based on CPM
   7.1.2.1 The difference between the suspected outlier and the result next closest in value must be greater than 10%.
   7.1.2.2 Remaining two results must be within at least 10% of each other.
7.1.3 Removal based on concentrations
   7.1.3.1 The difference between the suspected outlier and the result next closest in value must be greater than 15%.
   7.1.3.2 Remaining two results must be within at least 15% of each other.
      8.0 Assay Specifications

8.1 QC samples:
8.1.1 QC Nominal Value are as follows:
   8.1.1.1 QC1 (200 pg/mL)
   8.1.1.2 QC2 (500 pg/mL)
   8.1.1.3 QC3 (800 pg/mL)

8.1.2 QC results are to be collected, range to be determined after appropriate number of QCs are collected. Assays to be reviewed for acceptance.

Diagram #1
PYY RIA Standard:

| Tube | |
|---|---|
| designation | Concentration of Standard |
| A | 1280 pg/mL |
| B | 640 pg/mL |
| C | 320 pg/mL |
| D | 160 pg/mL |
| E | 80 pg/mL |
| F | 40 pg/mL |
| G | 20 pg/mL |
| H | 10 pg/mL |

| | |
|---|---|
| Attachment #1 | |
| | 0.6% TX-100 |
| Reagent: | 0.6% TX-100 |
| Materials: | Milli-Q Distilled Water |
| | TX-100 |
| Preparation: | 1) Measure 50 mL of Milli-Q Distilled Water |
| | 4) Add 300 μL of TX-100 using positive |
| | displacement pipet |
| | 5) Mix well. |
| Attachment #2 | |
| | ¹²⁵I-Peptide PYY Tracer |
| Reagent: | ¹²⁵I-Peptide PYY Tracer |
| Materials: | 1x RIA Buffer |
| | ¹²⁵I-Peptide PYY |
| Preparation: | 1) Reconstitute tracer with 1 mL of 1x RIA Buffer. |
| | 7) Measure the quantity of the tracer on the Gamma |
| | Counter. |
| | Transfer 10 μL of reconstituted tracer to a tube. |
| | Place it in a black rack for the Gamma Counter. |
| | 8) Place rack on the Gamma Counter with the Stop |
| | rack behind it. |
| | 9) Press ‘Start” to begin counting, then |
| | ‘E’ to view CPM results. |
| | 10) Determine amount of tracer (X μL) to prepare and |
| | RIAB (Y mL) needed as follows: |

$\begin{matrix}X μL = \frac{( 5 μL ) ( cpm value ) ( \# tubes + 10 )}{( cpm from stock solution )} \\ Y mL = ( 0.1 ) ( \# tubes + 10 )\end{matrix}$

11) Combine X μL of ¹²⁵I-Peptide YY with Y
mL of RIAB. Mix well.

### EXAMPLE 3

9.0 Introduction:

A radioimmunoassay was developed to measure the concentration of Human Peptide YY 3-36 (hPYY) in canine plasma. Samples are collected with anticoagulant (EDTA) and protease inhibitor (aprotinin) and frozen. The assay is a four day process. Samples, controls, and standards are extracted in alcohol and dried on Day 1. All samples are reconstituted and mixed with a polyclonal rabbit antiserum directed against hPYY on Day 2. Iodinated hPYY is added on Day 3. Specific precipitating agents (Goat anti-Rabbit IgG and Normal Rabbit Serum) are added on Day 4. Bound tracer is separated from free tracer by centrifugation, and the bound tracer is counted in the gamma counter. Concentration is calculated by interpolation of a standard curve and assay performance is controlled with Quality Control samples.

10.0 Materials:

2.1 Peninsula PYY kit (Peninsula Laboratories, Cat. No. S-2043-0001)

2.4 Reagent Alcohol (Fisher Inc., Cat. No. A995-4) (or equivalent)

2.7 Stripped Canine plasma (with EDTA, fasted, pooled) Golden West Biologics Inc. (Cat. No., ABP2020-E)

2.8 Ice Baths (Fisher, Cat No. 11-676-36) (or equivalent)

2.5 Disposable 10 mL pipets (Fisher Cat. No. 13-678-11E) (or equivalent)

2.10 Standard Synthetic Human PYY (3-36) (Bachem Cat. No. H8585)

2.11 Distilled Water (Milli-Q Millipore, Cat. No. ZMQ56VFT1) (or equivalent)

2.8 Triton X-100 (Sigma, Cat. No. T-9284) (or equivalent)

2.9 Aluminum Foil (Fisher, Cat. No. 01-213-3) (or equivalent)

2.13 Aprotinin (ICN Biomedicals Inc. Cat. No. 190779) (or equivalent)

2.14 12×75 mm tubes (Evergreen Scientific, Cat. No. 214-2023-010) (or equivalent)

2.15 12×75 mm tube caps (Evergreen Scientific, Cat. No. 300-2912-G20) (or equivalent)

2.16 1.5 mL microfuge tubes (Fisher, Cat. No. 05-402-25) (or equivalent)

11.0 Instruments:

1.1 Wallac WIZARD 1470 Automatic Gamma Counter (Perkin Elmer, Model No. 1470-002) (or equivalent)

1.2 Isotemp Basic Freezer, −70° C. (Kendro Laboratory Products, Model No. C90-3A31) (or equivalent)

1.3 CentriVap Concentrator (Labconco, Cat. No. 7810000) (or equivalent)

1.4 VX-2500 Multi-tube Vortexer (VWR, Cat. No. 58816-115) (or equivalent)

1.5 Marathon 21000R Centrifuge (Fisher, Cat. No. 04-977-21000R) (or equivalent)

1.6 Swinging bucket rotor (Fisher, Cat. No. 04-976-006) (or equivalent)

1.7 Motorized pipet-aid (Fisher, Cat. No. 13-681-15E) (or equivalent)

1.8 Eppendorf Micropipette
1.8.1 2 μL-20 μL (Fisher, Cat. No. 21-371-6) (or equivalent)
1.8.2 20 μL-200 μL (Fisher, Cat. No.21-371-10) (or equivalent)
1.8.3 100 μL-1000 μL (Fisher, Cat. No.21-371-13) (or equivalent)

1.9 Eppendorf Repeating Pipettor (Fisher, Cat. No. 21-380-9) (or equivalent)

1.10 Eppendorf Repeating Pipettor Combi-tips
1.10.1 2.5 mL (Fisher, Cat. No.21-381-331) (or equivalent)
1.10.2 25 mL (Fisher, Cat. No. 21-381-115) (or equivalent)

1.11 Positive displacement pipet (Fisher, Cat. No. 21-169-10A) (or equivalent)

12.0 Procedure

Day 1

4.1 Thaw necessary reagents and samples for the assay. Prepare RIA buffer to 1× concentration (RIAB) if sufficient amount is not available.

4.2 Prepare standard curve samples in pooled stripped canine plasma. Prepare as follows if using a starting concentration of 12.8 μg/mL.
4.2.1 Add 990 μL RIAB to tube O.
4.2.2 Add 990 μL pooled plasma to tube A.
4.2.3 Add 500 μL pooled plasma to tubes B-H.
4.2.4 Add 10 μL 12.8 μg/mL Standard to tube O. Vortex.
4.2.5 Add 10 μL solution from tube O to tube A. Vortex.
4.2.6 Add 500 μL solution from tube A to tube B. Vortex.
4.2.7 Add 500 μL solution from tube B to tube C. Vortex.
4.2.8 Repeat dilutions as in 4.2.7 through tube H. (See Diagram #1)

4.3 Dilute unknown canine plasma samples to be tested if necessary. Samples should be diluted in pooled stripped canine plasma. Each sample should have a final volume of 400 μL of neat or diluted sample.

4.4 Add 400 μL of pooled stripped canine plasma to NSB and TB tubes

4.5 Add 400 μL of each prepared Standard sample from 4.2.5 to 4.2.8 to respective standard curve tubes H-A (See Diagram #1).

4.6 Add 400 μL of QC samples to respective tubes.

4.7 Add 1.2 mL of cold alcohol to tubes containing NSB, TB, all Standards, QC samples, and canine plasma samples to be tested. Cap, Vortex.

4.8 Incubate all samples on ice for 30-60 minutes.

4.9 Turn on the cold-trap switch on the Concentrator.

4.10 Centrifuge all tubes at 3000 rpm, 4° C. for 15 minutes.

4.11 Transfer 1.3 mL of supernatant from each sample to a new set of empty tubes. Store in an ice bath or at 2-8° C. if not spun immediately.

4.12 Place samples in the Concentrator.

4.13 Samples should spin for two hours at 40° C., then at ambient temperature for a total of 5 hours or until dry.

4.14 Remove dried samples, cover and store overnight at 2-8° C.

Day 2

4.15 Remove the dried tubes from the 2-8° C. cooler.

4.16 Add 100 μL of 4× RIA buffer concentrate to each tube.

4.17 Add 100 μL of 0.6% TX100 to each tube. (Attachment #1) Vortex for a minimum of 30 seconds to ensure all extracts are fully reconstituted.

4.18 Incubate all samples on ice for 30-60 minutes.

4.19 Add 200 μL of distilled water to each tube. Vortex.

4.20 Transfer 100 μL of each sample extract to respective tube.
Note: NSB, TB, TC, Standard Curve samples, and QCs are typically run in triplicate, requiring three tubes per sample. Canine plasma samples many be tested in any variation (up to three replicates) depending on sample availability.

4.21 Prepare Rabbit anti-PYY as described in the Peninsula Laboratories kit insert.

4.22 Add 100 μL RIAB to each NSB tube.

4.23 Add 200 μL RIAB to each TC tube.

4.24 Add 100 μL Rabbit anti-PYY to all remaining tubes. Vortex.

4.25 Cover with foil and store overnight at 2-8° C.

Day 3

4.26 Remove the tubes from the 2-8° C. cooler.

4.27 Prepare ¹²⁵I-Peptide YY tracer (Attachment #2).

4.28 Add 100 μL of prepared tracer to all tubes. Cap and vortex.

4.29 Store overnight at 2-8° C.

Day 4

4.30 Remove the tubes from the 2-8° C. cooler.

4.31 Prepare Goat anti-Rabbit IgG serum (GARGG) and Normal Rabbit Serum (NRS) as described in the Peninsula Laboratories kit insert.

4.32 Add 100 μL GARGG to each tube (except TC tubes).

4.33 Add 100 μL NRS to each tube (except TC tubes). Vortex.

4.34 Incubate 90-120 minutes at room temperature.

4.35 Add 500 μL RIAB to tubes to be centrifuged immediately (except TC tubes). Vortex.
Note: 500 μL RIAB should be added to tubes just prior to centrifugation. Only add RIAB to the number of tubes that are ready to be centrifuged. 500 μL RIAB should be added to additional tubes when they are ready to be centrifuged.

4.36 Centrifuge tubes (containing 500 μL RIAB) at 3000 rpm at 4° C., for 15 minutes. Do not centrifuge TC tubes.

4.37 Aspirate supernatant from centrifuged tubes.

4.38 Place tubes in designated racks for counting on the Gamma counter and determine the CPM using standard techniques.

13.00 Evaluation of Results

5.1 The following guidelines are applied to the identification and rejection of outliers in the assay. In order for a result to qualify as an outlier and not be included in the final calculation of results, all of the following conditions must be met. Outliers can be removed based on CPM or concentration results.
5.1.1 There must be at least three results to evaluate.
5.1.2 Removal based on CPM
   5.1.2.1 The difference between the suspected outlier and the result next closest in value must be greater than 10%.
   5.1.2.2 The remaining two results must be within at least 10% of each other.
5.1.3 Removal based on concentrations
   5.1.3.1 The difference between the suspected outlier and the result next closest in value must be greater than 15%.
   5.1.3.2 The remaining two results must be within at least 15% of each other.
      14.0 Assay Specifications

5.2 QC Samples:
5.2.1 QC nominal values are as follows:
   5.2.1.1 C1 (200pg/mL)
   5.2.1.2 QC2 (500 pg/mL)
   5.2.1.3 QC3 (800 pg/mL)

5.2.2 QC results are to be collected, range to be determined after appropriate number of QCs are collected. Assays to be reviewed for acceptance.

Diagram #1
PYY RIA Standard:

| Tube | |
|---|---|
| designation | Concentration of Standard |
| A | 1280 pg/mL |
| B | 640 pg/mL |
| C | 320 pg/mL |
| D | 160 pg/mL |
| E | 80 pg/mL |
| F | 40 pg/mL |
| G | 20 pg/mL |
| H | 10 pg/mL |

| | |
|---|---|
| Attachment #1 | |
| | 0.6% TX-100 |
| Reagent: | 0.6% TX-100 |
| Materials: | Milli-Q Distilled Water |
| | TX-100 |
| Preparation: | 1) Measure 50 mL of Milli-Q Distilled Water |
| | 2) Add 300 μL of TX-100 using positive |
| | displacement pipet |
| | 3) Mix well. |
| Attachment #2 | |
| | ¹²⁵I-Peptide PYY Tracer |
| Reagent: | ¹²⁵I-Peptide PYY Tracer |
| Materials: | 1x RIA Buffer |
| | ¹²⁵I-Peptide PYY |
| Preparation: | 1) Reconstitute tracer with 1 mL of 1x RIA Buffer. |
| | 2) Measure the quantity of the tracer on the Gamma |
| | Counter. |
| | Transfer 10 μL of reconstituted tracer to a tube. |
| | Place it in a black rack for the Gamma Counter |
| | with Program #30 attached. |
| | 3) Place rack on the Gamma Counter with the Stop |
| | rack behind it. |
| | 4) Press ‘Start” to begin counting, then |
| | ‘E’ to view CPM results. |
| | 5) Determine amount of tracer (X μL) to prepare and |
| | RIAB (Y mL) needed as follows: |

$\begin{matrix}X μL = \frac{( 5 μL ) ( cpm value ) ( \# tubes + 10 )}{( cpm from stock solution )} \\ Y mL = ( 0.1 ) ( \# tubes + 10 )\end{matrix}$

6) Combine X μL of ¹²⁵I-Peptide YY with Y mL
of RIAB. Mix well.

### EXAMPLE 4

1.0 Introduction:

A radioimmunoassay was developed to measure the concentration of Human Peptide YY 3-36 (hPYY) in rabbit plasma. Samples are collected with anticoagulant (EDTA) and protease inhibitor (aprotinin) and frozen. The assay is a four day process. Samples, controls, and standards are extracted in alcohol and dried on Day 1. All samples are reconstituted and mixed with a polyclonal rabbit antiserum directed against hPYY on Day 2. Iodinated hPYY is added on Day 3. Specific precipitating agents (Goat anti-Rabbit IgG and Normal Rabbit Serum) are added on Day 4. Bound tracer is separated from free tracer by centrifugation, and the bound tracer is counted in the gamma counter. Concentration is calculated by interpolation of a standard curve and assay performance is controlled with Quality Control samples.

2.0 Materials:

2.1 Peninsula PYY kit (Peninsula Laboratories, Cat. No. S-2043-0001)

2.5 Reagent Alcohol (Fischer, Cat. No A995-4)(or equivalent)

2.9 Stripped Rabbit plasma (with EDTA, fasted, pooled) Golden West Biologics Inc. (Cat. No., ABP-2140E)

2.10 Ice Baths (Fisher, Cat No. 11-676-36) (or equivalent)

2.5 Disposable 10 mL pipets (Fisher Cat. No. 13-678-11E) (or equivalent)

2.12 Standard Synthetic Human PYY (3-36) (Bachem Cat. No. H8585)

2.13 Distilled Water (Milli-Q Millipore, Cat. No. ZMQ56VFT1) (or equivalent)

2.8 Triton X-100 (Sigma, Cat. No. T-9284) (or equivalent)

2.9 Aluminum Foil (Fisher, Cat. No. 01-213-3) (or equivalent)

2.17 Aprotinin (ICN Biomedicals Inc. Cat. No. 190779) (or equivalent)

2.18 12×75 mm tubes (Evergreen Scientific, Cat. No. 214-2023-010) (or equivalent)

2.19 12×75 mm tube caps (Evergreen Scientific, Cat. No. 300-2912-G20) (or equivalent)

2.20 1.5 mL microfuge tubes (Fisher, Cat. No. 05-402-25) (or equivalent)

3.0 Instruments:

3.1 Wallac WIZARD 1470 Automatic Gamma Counter (Perkin Elmer, Model No. 1470-002) (or equivalent)

3.2 Isotemp Basic Freezer, −70° C. (Kendro Laboratory Products, Model No. C90-3A31) (or equivalent)

3.3 CentriVap Concentrator (Labconco, Cat. No. 7810000) (or equivalent)

3.4 VX-2500 Multi-tube Vortexer (VWR, Cat. No. 58816-115) (or equivalent)

3.5 Marathon 21000R Centrifuge (Fisher, Cat. No. 04-977-21000R) (or equivalent)

3.6 Swinging bucket rotor (Fisher, Cat. No. 04-976-006) (or equivalent)

3.7 Motorized pipet-aid (Fisher, Cat. No. 13-681-15E) (or equivalent)

3.8 Eppendorf Micropipette
3.8.1 2 μL-20 μL (Fisher, Cat. No. 21-371-6) (or equivalent)
3.8.2 20 μL-200 μL (Fisher, Cat. No.21-371-10) (or equivalent)
3.8.3 100 μL-1000 μL (Fisher, Cat. No.21-371-13) (or equivalent)

3.9 Eppendorf Repeating Pipettor (Fisher, Cat. No. 21-380-9) (or equivalent) 3.10 Eppendorf Repeating Pipettor Combi-tips
3.10.1 2.5 mL (Fisher, Cat. No. 21-381-331) (or equivalent)
3.10.2 25 mL (Fisher, Cat. No. 21-381-115) (or equivalent)

3.11 Positive displacement pipet (Fisher, Cat. No. 21-169-10A) (or equivalent)

4.0 Procedure

Day 1

4.39 Thaw necessary reagents and samples for the assay. Prepare RIA buffer to 1× concentration (RIAB) if sufficient amount is not available.

4.40 Prepare standard curve samples in pooled stripped rabbit plasma. Prepare as follows if using a starting concentration of 12.8 μg/mL.
4.40.1 Add 990 μL RIAB to tube O.
4.40.2 Add 990 μL pooled plasma to tube A.
4.40.3 Add 500 μL pooled plasma to tubes B-H.
4.40.4 Add 10 μL 12.8 μg/mL Standard to tube O. Vortex.
4.40.5 Add 10 μL solution from tube O to tube A. Vortex.
4.40.6 Add 500 μL solution from tube A to tube B. Vortex.
4.40.7 Add 500 μL solution from tube B to tube C. Vortex.
4.40.8 Repeat dilutions as in 4.2.7 through tube H. (See Diagram #1)

4.41 Dilute unknown rabbit plasma samples to be tested if necessary. Samples should be diluted in pooled stripped rabbit plasma. Each sample should have a final volume of 400 μL of neat or diluted sample.

4.42 Add 400 μL of pooled stripped rabbit plasma to NSB and TB tubes.

4.43 Add 400 μL of each prepared Standard sample from 4.2.5 to 4.2.8 to respective standard curve tubes H-A (See Diagram #1).

4.44 Add 400 μL of QC samples to respective tubes.

4.45 Add 1.2 mL of cold alcohol to tubes containing NSB, TB, all Standards, QC samples, and rabbit plasma samples to be tested. Cap, vortex.

4.46 Incubate all samples on ice for 30-60 minutes.

4.47 Turn on the cold-trap switch on the Concentrator.

4.48 Centrifuge all tubes at 3000 rpm, 4° C. for 15 minutes.

4.49 Transfer 1.3 mL of supernatant from each sample to a new set of empty tubes. Store in an ice bath or at 2-8° C. if not spun immediately.

4.50 Place samples in the Concentrator.

4.51 Samples should spin for two hours at 40° C., then at ambient temperature for a total of 5 hours or until dry.

4.52 Remove dried samples, cover and store overnight at 2-8° C.

Day 2

4.53 Remove the dried tubes from the 2-8° C. cooler.

4.54 Add 100 μL of 4× RIA buffer concentrate to each tube.

4.55 Add 100 μL of 0.6% TX100 to each tube. (Attachment #1) Vortex for a minimum of 30 seconds to ensure all extracts are fully reconstituted.

4.56 Incubate all samples on ice for 30-60 minutes.

4.57 Add 200 μL of distilled water to each tube. Vortex.

4.58 Transfer 100 μL of each sample extract to respective tube.
Note: NSB, TB, TC, Standard Curve samples, and QCs are typically run in triplicate, requiring three tubes per sample. Rabbit plasma samples many be tested in any variation (up to three replicates) depending on sample availability.

4.59 Prepare Rabbit anti-PYY as described in the Peninsula Laboratories kit insert.

4.60 Add 100 μL RIAB to each NSB tube.

4.61 Add 200 μL RIAB to each TC tube.

4.62 Add 100 μL Rabbit anti-PYY to all remaining tubes. Vortex.

4.63 Cover with foil and store overnight at 2-8° C.

Day 3

4.64 Remove the tubes from the 2-8° C. cooler.

4.65 Prepare ¹²⁵I-Peptide YY tracer (Attachment #2).

4.66 Add 100 μL of prepared tracer to all tubes. Cap and vortex.

4.67 Store overnight at 2-8° C.

Day 4

4.68 Remove the tubes from the 2-8° C. cooler.

4.69 Prepare Goat anti-Rabbit IgG serum (GARGG) and Normal Rabbit Serum (NRS) as described in the Peninsula Laboratories kit insert.

4.70 Add 100 μL GARGG to each tube (except TC tubes).

4.71 Add 100 μL NRS to each tube (except TC tubes). Vortex.

4.72 Incubate 90-120 minutes at room temperature.

4.73 Add 500 μL RIAB to tubes to be centrifuged immediately (except TC tubes). Vortex.
Note: 500 μL RIAB should be added to tubes just prior to centrifugation. Only add RIAB to the number of tubes that are ready to be centrifuged. 500 μL RIAB should be added to additional tubes when they are ready to be centrifuged.

4.74 Centrifuge tubes (containing 500 μL RIAB) at 3000 rpm at 4° C., for 15 minutes. Do not centrifuge TC tubes.

4.75 Aspirate supernatant from centrifuged tubes.

4.76 Place tubes in designated black racks for counting on the Gamma counter. The first rack should have the appropriate Program number attached. All racks that follow should contain no program number. Samples should be added in the following order:
4.76.1 NSB tubes
4.76.2 TB tubes
4.76.3 TC tubes
4.76.4 Standard tubes (increasing concentration)
4.76.5 QC samples (3-4 concentrations)
4.76.6 Unknown rabbit samples
4.76.7 QC samples (3-4 concentrations)

4.77 Place an empty black rack with the Stop label attached after all samples to be counted.

4.78 Press ‘Start’ on the Gamma Counter keypad to start counting.

4.79 Press ‘E’ for enter on the Gamma Counter keypad to display CPM results.

5.0 Evaluation of Results

5.1 The following guidelines are applied to the identification and rejection of outliers in the assay. In order for a result to qualify as an outlier and not be included in the final calculation of results, all of the following conditions must be met.
5.1.1 QCs and unknown samples:
   5.1.1.1 % CV of all replicates must be great than 20%.
   5.1.1.2 There must be at least three results to evaluate.
   5.1.1.3 The difference between the suspected outlier and the result next closest in value must be greater than 20%. The difference between the high and low remaining results must be less than 20%.
5.1.2 Standard Curve samples:
5.1.3 % CV of all replicates much be great than 15%.
   5.1.3.1 There must be at least three results to evaluate.
   5.1.3.2 The difference between the suspected outlier and the result next closest in value must be greater than 15%.
   5.1.3.3 The difference between the high and low remaining results must be less than 15%.
      6.0 Assay Specifications

6.1 QC samples are prepared at the following concentrations. Two QC samples at each concentration are tested in an assay. Four of the six QC samples tested must be within the following ranges (±30% of nominal concentration). At least one of the two QCs tested at each concentration must be within the specified range for data to be acceptable.
6.1.1 QC1 (100 pg/mL) 70-130 pg/mL
6.1.2 QC2 (200 pg/mL) 140-260 pg/mL

6.1.3 QC3 (500 pg/mL) 350-650 pg/mL.

Diagram #1
PYY RIA Standard:

| Tube | |
|---|---|
| designation | Concentration of Standard |
| A | 1280 pg/mL |
| B | 640 pg/mL |
| C | 320 pg/mL |
| D | 160 pg/mL |
| E | 80 pg/mL |
| F | 40 pg/mL |
| G | 20 pg/mL |
| H | 10 pg/mL |

| | |
|---|---|
| Attachment #1 | |
| | 0.6% TX-100 |
| Reagent: | 0.6% TX-100 |
| Materials: | Milli-Q Distilled Water |
| | TX-100 |
| Preparation: | 1) Measure 50 mL of Milli-Q Distilled Water |
| | 2) Add 300 μL of TX-100 using positive |
| | displacement pipet |
| | 3) Mix well. |
| Attachment #2 | |
| | ¹²⁵I-Peptide PYY Tracer |
| Reagent: | ¹²⁵I-Peptide PYY Tracer |
| Materials: | 1x RIA Buffer |
| | ¹²⁵I-Peptide PYY |
| Preparation: | 1) Reconstitute tracer with 1 mL of 1x RIA Buffer. |
| | 2) Measure the quantity of the tracer on the Gamma |
| | Counter. |
| | Transfer 10 μL of reconstituted tracer to a tube. |
| | Place it in a black rack for the Gamma Counter |
| | with Program #30 attached. |
| | 3) Place rack on the Gamma Counter with the Stop |
| | rack behind it. |
| | 4) Press ‘Start” to begin counting, then |
| | ‘E’ to view CPM results. |
| | 5) Determine amount of tracer (X μL) to prepare and |
| | RIAB (Y mL) needed as follows: |

$\begin{matrix}X μL = \frac{( 5 μL ) ( cpm value ) ( \# tubes + 10 )}{( cpm from stock solution )} \\ Y mL = ( 0.1 ) ( \# tubes + 10 )\end{matrix}$

6) Combine X μL of ¹²⁵I-Peptide YY with Y mL
of RIAB. Mix well.

### EXAMPLE 5

### Procedure for Producing Plasma Stripped of PYY

1.0 Introduction

This procedure was developed to remove endogenous material from plasma. This yields a stripped plasma matrix that can be used as the diluent in plasma assays. The method is a modification of the procedure described by Grandt et al (1994) and involves adsorption of endogenous material onto the C18 packing of solid phase extraction (SPE) cartridges.

2.0 Materials

2.1 Sep-Pak 20 cc 5 g C18 cartridges (Waters, Cat. No. WAT036925)(or equivalent)

2.2 Methanol, HPLC grade (MeOH)(Fisher, Cat. No. A452-4)(or equivalent)

2.3 Milli Q grade water (Nastech) (or equivalent)

2.4 Mono-basic potassium phosphate 1.0M (Sigma, Cat. No. P-8709) (or equivalent)

2.5 Dibasic potassium phosphate 1.0M (Sigma, Cat. No. P-8584) (or equivalent)

2.6 15 ml polypropylene tubes (Falcon, Cat. No. 352097) (or equivalent)

2.7 Fasting, EDTA plasma (Golden West Biologics) (or equivalent)

3.0 Instruments

3.1 12-port solid phase extraction vacuum manifold (Fisher Cat. No. 11-131 -31)

3.2 Gast oil-less vacuum pump (Fisher, Cat. No. 01-092-29)

3.3 Trap System comprising two 2 L flasks (with side arm) connected with the appropriate Tygon tubing.

3.4 Centrifuge with swinging bucket rotor (Marathon 21000R)(or equivalent)

4.0 Procedure

4.1 Prepare 500 mL of 50 mM potassium phosphate buffer.
4.1.1 Combine 25 mL of 1.0M mono-basic potassium phosphate solution and 25 mL of 1.0M dibasic potassium phosphate solution.

4.1.2 Add 450 mL of distilled water to potassium phosphate solution from 4.1.1 for a final volume of 500 mL.
4.1.3 Determine pH of solution. Reading should be 7.0.

4.2 Thaw normal plasma to be stripped. Pool all lots or separate aliquots to be stripped.

4.3 Centrifuge plasma for 15 minutes at 3000 rpm. This will remove any clots that may be present.

4.4 Transfer plasma supernatant to a new tube or container.

4.5 Set up vacuum pump system with the vacuum manifold and trap flask.

4.6 Place cartridges on the top of the vacuum manifold. Maximum number of cartridges to use at one time is three due to high vacuum required for these larger cartridges.

4.7 To activate the cartridges, fill reservoirs with MeOH and slowly draw the solvent through the cartridges until approximately ¼ volume of MeOH is remaining above the cartridge packing.
Note: The speed at which any liquid is drawn through the cartridge is controlled by the vacuum and the valves on the vacuum manifold. These controls should be modified throughout the procedure to adjust speed of the liquid to ensure effective stripping of the plasma.

4.8 Turn off the vacuum to allow packing to soak in MeOH for 10 minutes.

4.9 Draw remaining MeOH through the cartridge leaving a thin layer of solvent above the packing to prevent the packing from drying out.

4.10 Wash packing by filling reservoir with water and slowing drawing through the cartridge.

4.11 Repeat 4.10 for two more water washes.

4.12 Wash cartridges with prepared phosphate buffer solution. Fill reservoirs with phosphate buffer solution and slowly draw liquid through the cartridges.

4.13 Fill reservoirs with normal plasma to be stripped.

4.14 Draw plasma through drop-wise at a rate of approximately 1 mL per minute until water has been displaced. The bottom of filter will change from white to a light brown color.

4.15 Place 15 mL collection tubes under each cartridge.

4.16 Continue drawing plasma through each cartridge at approximately 1 mL per minute by adding plasma to the cartridge as it is drawn through to the collection tube. Do not allow packing to dry at any point.

4.17 Strip a maximum of 75 mL of normal plasma with each cartridge.

5.0 Reference

Grandt D et al (1994). Two molecular forms of peptide YY (PYY) are abundant in human blood: characterization of a radioimmunoassay recognizing PYY 1-36 and 3-36. Regulatory Peptides, 51; 151-159.

### EXAMPLE 6

### Bioavailability and Bioequivalence of Reference and Test Formulations

Bioavailability is defined as the rate and extent to which the active ingredient or active moiety is absorbed from a drug product and becomes available at the site of action. For drug products that are not intended to be absorbed into the bloodstream, bioavailability may be assessed by measurements intended to reflect the rate and extent to which the active ingredient or active moiety becomes available at the site of action.

This definition focuses on the processes by which the active ingredients or moieties are released from an oral dosage form and move to the site of action.

From a pharmacokinetic perspective, BA data for a given formulation provide an estimate of the relative fraction of the orally administered dose that is absorbed into the systemic circulation when compared to the BA data for a solution, suspension, or intravenous dosage form. In addition, BA studies provide other useful pharmacokinetic information related to distribution, elimination, the effects of nutrients on absorption of the drug, dose proportionality, linearity in pharmacokinetics of the active moieties and, where appropriate, inactive moieties. BA data may also provide information indirectly about the properties of a drug substance before entry into the systemic circulation, such as permeability and the influence of presystemic enzymes and/or transporters (e.g., p-glycoprotein).

Bioequivalence is defined as the absence of a significant difference in the rate and extent to which the active ingredient or active moiety in pharmaceutical equivalents or pharmaceutical alternatives becomes available at the site of drug action when administered at the same molar dose under similar conditions in an appropriately designed study.

As noted, both BE and product quality BA focus on the release of a drug substance from a drug product and subsequent absorption into the systemic circulation.

For both replicate and nonreplicate, in vivo pharmacokinetic BE studies, the following general approaches are recommended, recognizing that the elements may be adjusted for certain drug substances and drug products.

Study conduct:
The test or reference products should be administered with about 8 ounces (240 milliliters) of water to an appropriate number of subjects under fasting conditions, unless the study is a food-effect BA and BE study.
Generally, the highest marketed strength should be administered as a single unit. If warranted for analytical reasons, multiple units of the highest strength can be administered, providing the total single-dose remains within the labeled dose range.
An adequate washout period (e.g., more than 5 half lives of the moieties to be measured) should separate each treatment.
The drug content of the test product should not differ from that of the reference product by more than 5 percent.
Before and during each study phase, subjects should (1) be allowed water as desired except for 1 hour before and after drug administration, (2) be provided standard meals no less than 4 hours after drug administration, and (3) abstain from alcohol for 24 hours before each study period and until after the last sample from each period is collected.

Sample collection and sampling times:
Under normal circumstances, blood should be used and Y2 Receptor Binding compounds should be extracted from blood by the methods of the instant case. Blood samples should be drawn at appropriate times to describe the absorption, distribution, and elimination phases of the drug. For most drugs, 12 to 18 samples, including a predose sample, should be collected per subject per dose. This sampling should continue for at least three or more terminal half lives of the drug. The exact timing for sample collection depends on the nature of the drug and the input from the administered dosage form. The sample collection should be spaced in such a way that the maximum concentration of the drug in the blood (Cmax) and terminal elimination rate constant (_z) can be estimated accurately. At least three to four samples should be obtained during the terminal log-linear phase to obtain an accurate estimate of _z from linear regression. The actual clock time when samples are drawn as well as the elapsed time related to drug administration should be recorded.

Subjects with predose plasma concentrations:
If the predose concentration is less than or equal to 5 percent of Cmax value in that subject, the subject's data without any adjustments can be included in all pharmacokinetic measurements and calculations. If the predose value is greater than 5 percent of Cmax, the subject should be dropped from all BE study evaluations.

The following pharmacokinetic information is recommended:
Plasma concentrations and time points
Subject, period, sequence, treatment
AUC₀₋ₜ, AUC₀₋_{_{—}}, Cmax, Tmax, _z, and t_{1/2}
Intersubject, intrasubject, and/or total variability, if available
Cmin (concentration at the end of a dosing interval), Cav (average concentration during a dosing interval), degree of fluctuation [(Cmax-Cmin)/Cav], and swing [(Cmax-Cmin)/Cmin] if steady-state studies are employed
Partial AUC, requested only as discussed in section III. A.9.a.

In addition, the following statistical information should be provided for AUC₀₋ₜ, AUC₀₋_{_{—}}, and Cmax:
Geometric mean
Arithmetic mean
Ratio of means
Confidence intervals

Logarithmic transformation should be provided for measures used for BE demonstration. Rounding off of confidence interval values:
Confidence interval (CI) values should not be rounded off; therefore, to pass a CI limit of 80 to 125, the value should be at least 80.00 and not more than 125.00.

Bioequivalence is generally indicated when the test and reference formulations have confidence intervals for pharmacokinetic parameters, especially Cmax and AUC, from 70 to 130 and preferredly from 80 to 125.

1. A method for determining an amount of Peptide YY in a sample or aliquot of blood comprising:
obtaining an aliquot of blood;
extracting the PYY from matrix in blood to which the peptide may be bound such bound PYY is free released in solution; and
measuring the amount of PYY in solution.

2. The method of claim 1 wherein the PYY is extracted from the matrix by means of precipitation.

3. The method of claim 2 wherein the matrix is precipitated by an agent selected from the group consisting of a salt, an organic solvent, an acid and an organic polymer.

4. The method of claim 3 wherein the organic solvent is selected from the group consisting of ethanol, methanol, propanol and acetone.

5. The method of claim 4 wherein the organic solvent is ethanol.

6. The method of claim 1 wherein the amount of PYY present in the aliquot or sample of blood is determined using an immunoassay.

7. The method of claim 6 wherein the immunoassay is a radioimmunoassay.

8. The method of claim 1 further comprising removing cellular components from the blood sample prior to extracting the PYY from the matrix so as to produce an aliquot of plasma, and extracting the PYY bound to matrix in the plasma and determining the amount of PYY present in the plasma.

9. The method of claim 8 wherein the amount of PYY present in the plasma is determined using an immunoassay.

10. The method of claim 9 wherein the immunoassay is a radioimmunoassay.

11. A method for determining the total amount of PYY present in an aliquot of blood comprising:
removing cellular components in the aliquot of blood to produce an aliquot of plasma
extracting PYY from a proteinaceous matrix in the plasma; and
measuring the amount of PYY present in the plasma.

12. The method of claim 11 wherein the amount of PYY present in the plasma is determined using an immunoassay.

13. The method of claim 11 wherein the matrix is precipitated by an agent selected from the group consisting of a salt, an organic solvent, an acid and an organic polymer.

14. The method of claim 13 wherein the organic solvent is selected from the group consisting of ethanol, methanol, propanol and acetone.

15. A method of comparing formulations to establish bioequivalence of a reference formulation and a test formulation of a PYY comprising the steps of:
a. administering the reference or a test formulation to a group of subjects and obtaining blood samples at intervals after said administration;
b. obtaining a solvent extracted plasma sample from said blood samples;
c. measuring the concentration of PYY in said samples and determining pharmacokinetic parameters, including Cmax and AUC, in each of the samples of plasma; and
d. comparing the test and reference formulation pharmacokinetic parameters to determine bioequivalency.

16. The method of claim 15 wherein said the PYY is administered intranasally.

17. The method of claim 15 wherein an organic solvent miscible in water is added to the blood samples under conditions wherein PYY bound to other components of the blood is freed and proteinaceous material present in the blood samples precipitates leaving the PYY free in solution.

18. The method of claim 15 wherein the amount of PYY present in solution is determined by an immunoassay.


SEQUENCE LISTING (40 sequences)

SEQ ID NO: 1 - protein, 36 aa, Homo sapiens
YPIKPEAPGEDASPEELNRYYASLRHYLNLVTRQRY

SEQ ID NO: 2 - protein, 34 aa, Homo sapiens
IKPEAPGEDASPEELNRYYASLRHYLNLVTRQRY

SEQ ID NO: 3 - protein, 15 aa, Homo sapiens
ASLRHYLNLVTRQRY

SEQ ID NO: 4 - protein, 33 aa, Homo sapiens
KPEAPGEDASPEELNRYYASLRHYLNLVTRQRY

SEQ ID NO: 5 - protein, 32 aa, Homo sapiens
PEAPGEDASPEELNRYYASLRHYLNLVTRQRY

SEQ ID NO: 6 - protein, 31 aa, Homo sapiens
EAPGEDASPEELNRYYASLRHYLNLVTRQRY

SEQ ID NO: 7 - protein, 30 aa, Homo sapiens
APGEDASPEELNRYYASLRHYLNLVTRQRY

SEQ ID NO: 8 - protein, 29 aa, Homo sapiens
PGEDASPEELNRYYASLRHYLNLVTRQRY

SEQ ID NO: 9 - protein, 28 aa, Homo sapiens
GEDASPEELNRYYASLRHYLNLVTRQRY

SEQ ID NO: 10 - protein, 27 aa, Homo sapiens
EDASPEELNRYYASLRHYLNLVTRQRY

SEQ ID NO: 11 - protein, 26 aa, Homo sapiens
DASPEELNRYYASLRHYLNLVTRQRY

SEQ ID NO: 12 - protein, 25 aa, Homo sapiens
ASPEELNRYYASLRHYLNLVTRQRY

SEQ ID NO: 13 - protein, 24 aa, Homo sapiens
SPEELNRYYASLRHYLNLVTRQRY

SEQ ID NO: 14 - protein, 23 aa, Homo sapiens
PEELNRYYASLRHYLNLVTRQRY

SEQ ID NO: 15 - protein, 22 aa, Homo sapiens
EELNRYYASLRHYLNLVTRQRY

SEQ ID NO: 16 - protein, 21 aa, Homo sapiens
ELNRYYASLRHYLNLVTRQRY

SEQ ID NO: 17 - protein, 20 aa, Homo sapiens
LNRYYASLRHYLNLVTRQRY

SEQ ID NO: 18 - protein, 19 aa, Homo sapiens
NRYYASLRHYLNLVTRQRY

SEQ ID NO: 19 - protein, 18 aa, Homo sapiens
RYYASLRHYLNLVTRQRY

SEQ ID NO: 20 - protein, 17 aa, Homo sapiens
YYASLRHYLNLVTRQRY

SEQ ID NO: 21 - protein, 16 aa, Homo sapiens
YASLRHYLNLVTRQRY

SEQ ID NO: 22 - protein, 22 aa, Homo sapiens
DELNRYYASLRHYLNLVTRQRY

SEQ ID NO: 23 - protein, 24 aa, Homo sapiens
TPEELNRYYASLRHYLNLVTRQRY

SEQ ID NO: 24 - protein, 25 aa, Homo sapiens
VSPEELNRYYASLRHYLNLVTRQRY

SEQ ID NO: 25 - protein, 26 aa, Homo sapiens
EASPEELNRYYASLRHYLNLVTRQRY

SEQ ID NO: 26 - protein, 27 aa, Homo sapiens
DDASPEELNRYYASLRHYLNLVTRQRY

SEQ ID NO: 27 - protein, 30 aa, Homo sapiens
VPGEDASPEELNRYYASLRHYLNLVTRQRY

SEQ ID NO: 28 - protein, 31 aa, Homo sapiens
DAPGEDASPEELNRYYASLRHYLNLVTRQRY

SEQ ID NO: 29 - protein, 33 aa, Homo sapiens
QPEAPGEDASPEELNRYYASLRHYLNLVTRQRY

SEQ ID NO: 30 - protein, 33 aa, Homo sapiens
RPEAPGEDASPEELNRYYASLRHYLNLVTRQRY

SEQ ID NO: 31 - protein, 33 aa, Homo sapiens
NPEAPGEDASPEELNRYYASLRHYLNLVTRQRY

SEQ ID NO: 32 - protein, 34 aa, Homo sapiens
VKPEAPGEDASPEELNRYYASLRHYLNLVTRQRY

SEQ ID NO: 33 - protein, 34 aa, Homo sapiens
LKPEAPGEDASPEELNRYYASLRHYLNLVTRQRY

SEQ ID NO: 34 - protein, 27 aa, Homo sapiens
DDASPDELNRYYASLRHYLNLVTRQRY

SEQ ID NO: 35 - protein, 31 aa, Homo sapiens
DAPGEDATPEELNRYYASLRHYLNLVTRQRY

SEQ ID NO: 36 - protein, 33 aa, Homo sapiens
NPEAPGEDASPDELNRYYASLRHYLNLVTRQRY

SEQ ID NO: 37 - protein, 34 aa, Homo sapiens
LKPEAPGDDASPEELNRYYASLRHYLNLVTRQRY

SEQ ID NO: 38 - protein, 36 aa, Rat
YPAKPEAPGEDASPEELSRYYASLRHYLNLVTRQRY

SEQ ID NO: 39 - protein, 36 aa, Pig; identical to SEQ ID NO: 38

SEQ ID NO: 40 - protein, 36 aa, guinea pig
YPSKPEAPGSDASPEELARYYASLRHYLNLVTRQRY